(19) United States
(12) Patent Application Publication
(10) Pub. No.: US 2007/0178578 A1
(43) Pub. Date: Aug. 2, 2007

(54) Biofiltration system for treating airborne volatile organic compounds

(75) Inventors: Paul David Chalmer, Chelsea, MI (US); Abdul Shaheed Abdul, Troy, MI (US)

Correspondence Address: Jerome R. Drouillard, 10213 Tims Lake Blvd., Grass Lake, MI, 49240, US

(73) Assignee: NESA & Associates, Inc.

(21) Appl. No.: 11/341,299

(22) Filed: Jan. 27, 2006

(51) Int. Cl. A61L 9/01; C12M 1/14

(52) U.S. Cl. 435/266; 435/299.1

(57) ABSTRACT

A biofiltration system for treating biodegradable airborne contaminants such as VOCS includes a bio-reactor chamber with a sealed housing, and a heater for maintaining a temperature within the chamber. Several fabric-based bio-reactor panels are mounted within the chamber and are kept in a dampened state by means of an applicator system which applies a temperature-controlled liquid mixture containing microbes and nutrients to the bio-reactor panels. An air handling system moves air laden with biodegradable contaminants through the chamber and over the surfaces of the bio-reactor panels.

### BACKGROUND OF THE INVENTION

1. Field of the Invention

The present invention relates to a system for using biological agents for removing volatile organic compounds, such as paint solvents, from air.

2. Disclosure Information

Volatile organic compounds (“VOCS”) are organic compounds that easily become vaporized or gasified. As well as carbon, VOCS typically contain elements such as fluorine, chlorine, bromine, oxygen, hydrogen, sulfur and nitrogen. VOCS arise from burning of fuels, as well as from use of and handling of paints and other coatings, solvents, household chemicals, adhesives, and other types of chemicals. Common VOCS include benzene, formaldehyde, toluene, xylene, tetrachloroethylene, petroleum products such as gasoline, jet fuel, diesel fuel, and kerosene, and industrial solvents. VOCS are classified as an air pollutant, and their discharge into the atmosphere is limited by law and regulation.

Biofilter devices for VOCS are known. Such devices fall generally into four classes, namely bioscrubbers, bio-trickling filters, natural media biofilters and synthetic media biofilters. A common type of filter is a natural media biofilter, which has a column of soil, peat, compost, or bark. Such filters suffer from the problem that the compost or bark can become solidified and riddled with cracks, which reduce the efficiency of the biofilter. It is also difficult to maintain the operating temperature at a desired level with known biofilters. A system and method according to the present invention overcomes problems with known biofilters and provides effective biofiltration at low cost and with high robustness and reliability.

### SUMMARY OF THE INVENTION

A biofiltration system for treating biodegradable airborne contaminants includes a bio-reactor chamber with a sealed housing and a heater for maintaining the temperature within the chamber within a predetermined range. A number of fabric-based bio-reactor panels are mounted within the chamber, preferably in a generally vertical orientation. An applicator system applies a liquid mixture containing microbes and nutrients to the bio-reactor panels. The generally vertical orientation of the bio-reactor panels allows gravitational force to assist the nutrient application process. The applicator system preferably includes a reservoir located within the housing and a pump for circulating the liquid mixture containing microbes and nutrients from the reservoir. A heater receives the circulating liquid microbe and nutrient mixture. A distribution network receives liquid flowing from the heater and distributes it to the reactor panels. To facilitate this, the distribution network is arranged with a number of spray bars to deposit the liquid mixture upon an upper portion of the bio-reactor panels.

Because the present biofiltration system is intended to be used with air contaminated with VOCS, an air handling system is needed to move air laden with biodegradable contaminants through the chamber and over the surfaces of the bio-reactor panels. Such an air handling system preferably includes an air inlet and an air outlet extending through the housing, and a vacuum blower for drawing air from the chamber from the inlet to the outlet. An air inlet distribution manifold located within the lower portion of the chamber assures that the flowing air does not “short circuit” between the inlet and outlet of the sealed chamber.

It is desirable to keep the interior of the chamber at about 90° F.-110° F. for maximum microbial activity and growth, and this is achieved by using a fluid heater and pump for circulating heated fluid such as water or another aqueous solution through a heat exchanger mounted in a serpentine fashion within the chamber.

The sizing of a biofiltration system according to present invention to a source of VOCS may be materially assisted in some cases by the use of a VOC storage buffer positioned between the bio-reactor chamber and the source of VOCS. The storage buffer may, for example, comprise an activated carbon or zeolite adsorber which receives VOCS whenever the source is in operation and stores the VOCS for subsequent desorption and treatment by the present inventive biofiltration system.

According to another aspect of the present invention, a method for operating a biofiltration system for treating airborne VOCS includes the steps of defining a plurality of airflow passages extending between pairs of facing fabric bio-reactor panels contained within a sealed housing, and heating the bio-reactor panels to a temperature suited to promote the growth of VOC-consuming microbes. Thereafter, a liquid mixture containing microbes and nutrients is applied to the bio-reactor panels using a recirculation system. Then, air laden with VOCS is passed through the housing such that the VOC-laden air impinges upon the bio-reactor panels while flowing through the airflow passages such that microbes carried upon the bio-reactor panels will reduce the amount of VOCS within the air moving through the housing. As a further step, treated air leaving the reaction chamber of the biofiltration system may be passed through a post-treatment chamber for halting further microbial action within the treated air.

It is an advantage of the present biofiltration system that high VOC conversion efficiency may be achieved without the maintenance issues associated with organic packed bed bio-converters.

It is a further advantage of the present biofiltration system that the system is readily scaleable for use with VOC sources of different magnitudes.

It is a further advantage of the present biofiltration system that the operating temperature of the system is readily controllable to achieve high conversion efficiency.

It is a further advantage of the present biofiltration system that the operating humidity of the system is readily controllable to achieve high conversion efficiency.

It is a further advantage of the present biofiltration system that an optimum level of nutrients and microbes may be maintained throughout the reactive biofilter material.

It is a further advantage of the present biofiltration system that bioreactor panels are very stable and offer an excellent structure for VOC conversion.

It is a further advantage of the present biofiltration system that the package volume or “footprint” of the inventive system is smaller than known systems.

Other advantages, as well as features and objects of the present invention will become apparent to the reader of this specification.

### BRIEF DESCRIPTION OF THE DRAWINGS

FIG. 1 is a perspective view of a biofiltration system according to the present invention, showing with particularity several bio-reactor panels within the system's housing or reaction chamber.

FIG. 2 illustrates details of a heating system incorporated within a biofiltration device according to the present invention.

FIG. 3 shows additional details of the heating system and also shows a plan view of several bio-reactor panels according to the present invention.

FIG. 4 shows details of an air-handling system for moving air laden with biodegradable contaminants through the reaction chamber of the present device.

FIG. 5 is a block diagram showing placement of a VOC storage buffer and post-treatment chamber according to additional aspects of the present invention.

### DETAILED DESCRIPTION OF THE PREFERRED EMBODIMENTS

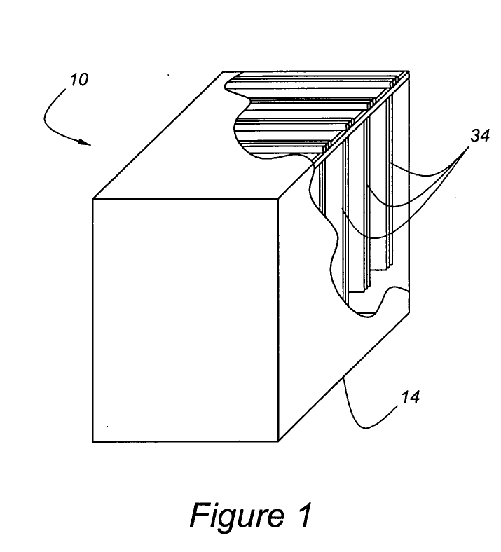

FIG. 1 shows bio-reactor 10 including sealed housing 14. As a matter of scale, the present inventors have determined that a bio-reactor constructed according to their invention and having an interior volume of about 36 cubic feet will handle approximately 100-150 SCFM of VOC-laden air from a typical paint booth. In order to accomplish this, bio-reactor 10 has eighteen single-plane bio-reactor panels, 34, which are fabricated from woven material similar to cotton terrycloth toweling. Bio-reactor panels 34 are hung vertically within sealed housing 14.

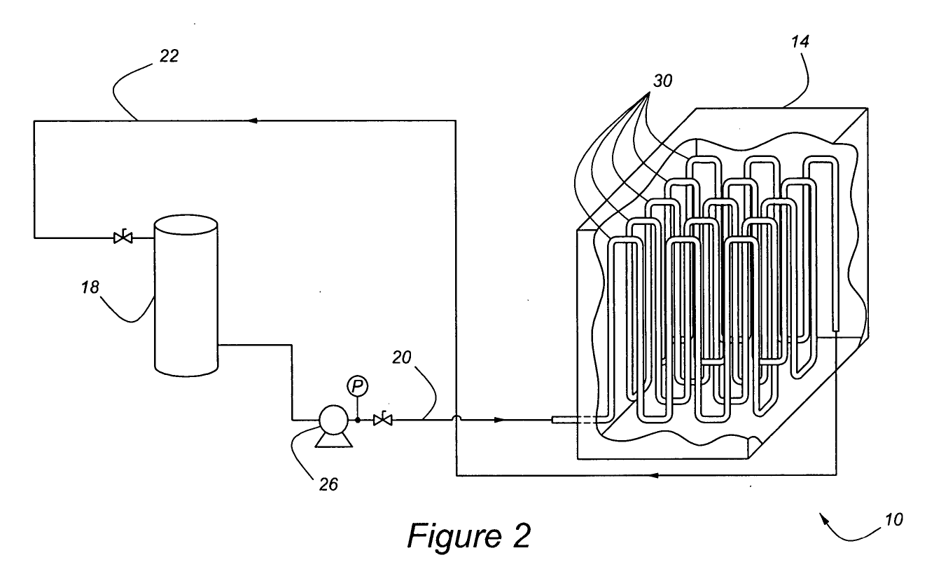

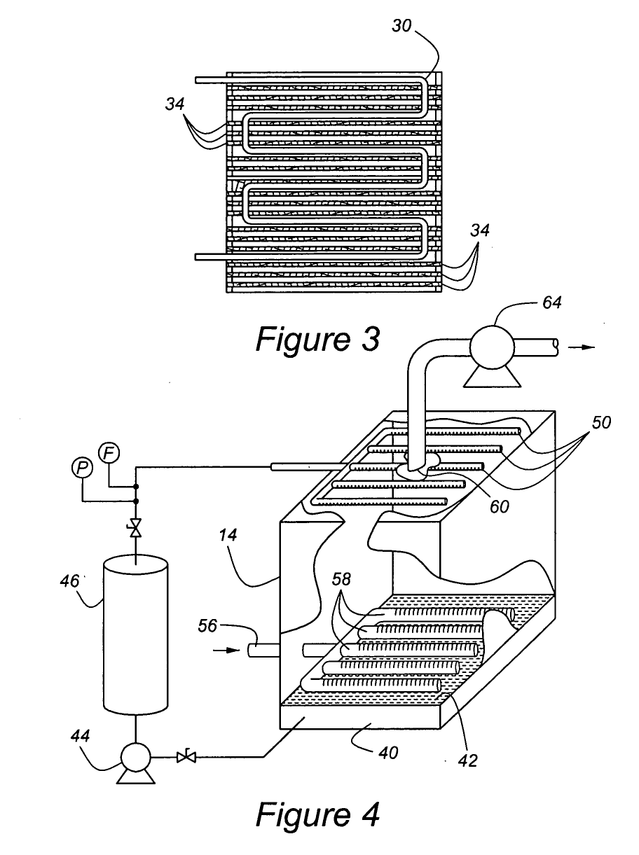

The temperature within housing 14 is maintained in part by the apparatus shown in FIG. 2, including heater 18, which receives a fluid such as water, or another heat transfer fluid known to those skilled in the art and suggested by this disclosure, from discharge line 22. Heated fluid is drawn from heater 18 by heater pump 26 and pushed through supply line 20 to heat exchanger 30 located within housing 14. The present inventors have determined that microbial growth and activity is optimized if the interior of housing 14 is maintained at about 90° F.-110° F. Serpentine heat exchanger 30 functions well in this regard by extending, as shown in FIG. 3, between successive groups of bio-reactor panels 34. The ability to precisely control the temperature within housing 14 gives the present biofiltration system a significant advantage, as compared with known bioreactor systems. Control of the temperature within chamber 14 is further achieved by controlling the temperature of the microbe-nutrient solution which is circulated upon bio-reactor panels 34. In general, temperature control is particularly needed because of the evaporative cooling which occurs as air flows through chamber 14. Those skilled in the art will appreciate in view of this disclosure that other heating systems may be used with a bio-reactor according to the present invention. For example, radiant heating or microwave heating could be used.

In order for the microbial reaction to function within housing 14 in an effective manner, it is necessary that a liquid mixture, 42, containing microbes and nutrients be continually reapplied to bio-reactor panels 34. This is accomplished through an applicator system (FIG. 4) which includes reservoir 40 located within a lower portion of housing 14. Nutrient pump 44 picks up microbe-laden nutrient solution from reservoir 40 and circulates the solution through a heater, 46, and then to spraybars 50 which are located in an upper region of housing 14. Spraybars 50 allow the nutrient-and-microbe-rich solution to drip onto bio-reactor panels 34, thereby keeping bio-reactor panels 34 moistened with the solution. Direct control of the temperature of mixture 42 assists materially in maintaining a high level of microbial activity. Nutrients may be provided by commercially available nitrate/phosphate preparations, one of which is manufactured by Spectrum Brands and sold under the trade name Peters Plant Food.

FIG. 4 also shows the air circulation system of the present device. Air is picked up from a source of VOCS, such as a paint booth or a VOC storage device, and delivered to inlet 56, wherein the air transitions to an inlet distribution manifold or diffuser 58. The purpose of manifold 58 is to assure that the air does not “short circuit” or go in a small column from inlet 56 to outlet 60. Air drawn by vacuum blower 64 through outlet 60 first moves upwardly through chamber 14 and flows over the surfaces of bio-reactor panels 34. In this manner the air is effectively treated and the VOCS are reduced by microbial action.

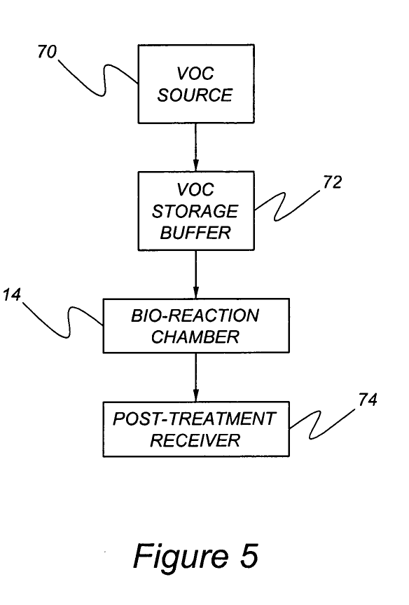

According to another aspect of the present invention, a method for operating a biofiltration system for treating airborne VOCS includes defining a plurality of airflow passages which extend between bio-reactor panels 34 and using heater 46 as well as heater 18 to maintain temperature within the bio-reactor panels and the chamber itself at about 90° F.-110° F. so as to promote the growth of the VOC-consuming microbes. The growth of the microbes is also promoted by applying a liquid nutrient and microbe-containing mixture to the bio-reactor panels using the previously described circulatory system. The method further includes the passing of air laden with VOCS through the housing such that the VOC laden air impinges upon the bio-reactor panels. The present method may further include passing treated air leaving housing 14 through a post-treatment receiver, 74, shown in FIG. 5, wherein microbial action would be halted. This may be accomplished, for example, by bubbling the post-treated air through a 1-2% hydrogen peroxide bath. This would assure that no viable microbes escape the treatment device.

FIG. 5, shows that the present VOC treatment device may be situated downstream from a VOC source 70 and between VOC storage buffer 72 and post-treatment receiver 74. In this manner, a VOC source 70, having a periodically high, or discontinuous, flow rate may be accommodated on a batch-processing basis by a relatively smaller-sized bio-reactor 14 by storing VOCS in storage buffer 72 and by operating bio-reactor 14 on a continuous basis.

Although the present invention has been described in connection with particular embodiments thereof, it is to be understood that various modifications, alterations, and adaptations may be made by those skilled in the art without departing from the spirit and scope of the invention set forth in the following claims. For example, the parameter values for temperature, flow rates, biofilter volume, and other parameters will be determined and controlled according to the requirements of a particular system constructed according to the present invention.

What is claimed:

1. A biofiltration system for treating biodegradable airborne contaminants, comprising:
a bio-reactor chamber comprising a sealed housing;
a heating system for maintaining the temperature within said chamber within a predetermined range;
a plurality of bio-reactor surfaces mounted within said chamber; and
an air handling system for moving air laden with biodegradable contaminants through said chamber and over the surfaces of said bio-reactor surfaces.

2. A biofiltration system for treating biodegradable airborne contaminants according to claim 1, wherein said heating system comprises a first system for controllably supplying heated fluid to a heat exchanger located within said sealed housing, and a second system for controlling the temperature of a liquid mixture containing microbes and nutrients delivered to said bio-reactor surfaces.

3. A biofiltration system for treating biodegradable airborne contaminants, comprising:
a bio-reactor chamber comprising a sealed housing;
a heater for maintaining the temperature within said chamber within a predetermined range;
a plurality of bio-reactor panels mounted within said chamber;
an applicator system for applying a liquid mixture containing microbes and nutrients to said bio-reactor panels; and
an air handling system for moving air laden with biodegradable contaminants through said chamber and over the surfaces of said bio-reactor panels.

4. A biofiltration system according to claim 3, wherein said heater comprises a heat exchanger mounted within said chamber and a fluid heater and a pump for circulating heated fluid through said heat exchanger.

5. A biofiltration system according to claim 3, wherein said heater maintains the temperature within said chamber in the range of 90° F.-110° F.

6. A biofiltration system according to claim 3, wherein said fluid comprises an aqueous solution.

7. A biofiltration system according to claim 3, wherein said air handling system comprises an air inlet and an air outlet extending through said housing, with said air handling system further comprising a vacuum blower for drawing air through said chamber from said air inlet to said air outlet.

8. A biofiltration system according to claim 3, wherein said air handling system further comprises an air inlet distribution manifold located within a lower portion of said chamber.

9. A biofiltration system according to claim 3, wherein said bioreactor panels are mounted vertically within said chamber.

10. A biofiltration system according to claim 3, wherein said applicator system comprises:
a reservoir located within said housing;
a pump for circulating the liquid mixture containing microbes and nutrients from said reservoir;
a heater for receiving the circulating liquid microbe and nutrient mixture and for warming said circulating liquid; and
a distribution network for receiving said circulating liquid from said heater, with said distribution network being arranged to deposit the liquid mixture upon said bio-reactor panels.

11. A biofiltration system according to claim 3, wherein said bio-reactor panels each comprise at least one woven fabric plane suspended vertically from an upper portion of said housing.

12. A biofiltration system for treating airborne volatile organic compounds (VOCS), comprising:
a bio-reactor chamber comprising a sealed housing;
a heater for maintaining the temperature within said chamber within a predetermined optimum range;
a plurality of fabric-based bio-reactor panels mounted within said chamber;
a system for applying a temperature-controlled liquid mixture containing microbes and nutrients to said bio-reactor panels; and
an air handling system for moving air laden with VOC through said chamber and over the surfaces of said bio-reactor panels.

13. A biofiltration system according to claim 12, further comprising a VOC storage buffer located between said bio-reactor chamber and a source of airborne VOCS.

14. A biofiltration system according to claim 12, wherein said VOC storage buffer comprises an activated carbon adsorber.

15. A biofiltration system according to claim 12, wherein said VOC storage buffer comprises a zeolite adsorber.

16. A biofiltration system according to claim 12, further comprising a post-treatment chamber for receiving treated air from said bio-reactor chamber and for stopping further microbial action within the treated air.

17. A biofiltration system according to claim 16, wherein said post-treatment chamber comprises a receiver for reacting said treated air with hydrogen peroxide.

18. A method for operating a biofiltration system for treating airborne volatile organic compounds (VOCS), comprising the steps of:
defining a plurality of airflow passages extending between bio-reactor panels contained within a sealed housing;
heating the interior of said sealed housing, including said bio-reactor panels, to a temperature suited to promote the growth of VOC-consuming microbes;
applying a liquid mixture containing microbes and nutrients to said bio-reactor panels using a recirculation system; and
passing air laden with VOCS through said housing such that said VOC-laden air impinges upon said bio-reactor panels while flowing through said airflow passages, such that microbes carried upon said bio-reactor panels will reduce the amount of VOCS within the air moving through the housing.

19. A method according to claim 18, further comprising the step of passing treated air leaving said housing through a post-treatment receiver for stopping further microbial action within the treated air.

20. A method according to claim 18, further comprising the step of directly controlling the temperature of said liquid mixture containing microbes and nutrients.
